(19) United States
(12) Patent Application Publication
(10) Pub. No.: US 2026/0007315 A1
(43) Pub. Date: Jan. 8, 2026

(54) ARTIFICIAL-INTELLIGENCE HEART MONITORING MODULE ADAPTIVE TO SPHYGMOMANOMETER

(71) Applicant: Kuo-Yuan Chang, New Taipei City (TW)

(72) Inventor: Kuo-Yuan Chang, New Taipei City (TW)

(21) Appl. No.: 18/821,769

(22) Filed: Aug. 30, 2024

(30) Foreign Application Priority Data
Jul. 5, 2024 (TW) 113125375

(51) Int. Cl. A61B 5/021; A61B 5/00; A61B 5/022

(52) U.S. Cl. CPC A61B 5/02108; A61B 5/02233; A61B 5/7264

(57) ABSTRACT

An AI heart monitoring module adaptive to sphygmomanometer is provided, including a housing with a first connection port and a second connection port; a pressure sensing unit disposed inside the housing for sensing pressure pulses of gas flowing through the first connection port and the second connection port to generate analog data of the pressure pulses; a processing unit disposed inside the housing, electrically connected with the pressure sensing unit, receiving the analog data of the pressure pulses, and converting the analog data of the pressure pulses into digital data of the pressure pulses, and processing the digital data with AI; a transmission unit disposed inside the housing, electrically connected with the processing unit, and outputting all or a portion of the digital data of the pressure pulses to an external device; and an indication unit disposed on the housing, electrically connected with the transmission unit, and responding to an event that the transmission unit completes outputting the digital data of the pressure pulses to the external device.

### 1. FIELD OF THE INVENTION

The present invention relates to a wireless heart monitoring module adaptive to sphygmomanometer, particularly to a heart disease monitoring module that can be added to a cuff-type sphygmomanometer.

### 2. DESCRIPTION OF THE PRIOR ART

The traditional cuff-type sphygmomanometer normally functions as a single and independent device to measure physiological information, such as blood pressures and heartbeats. The measured blood pressures and heartbeats may be stored in the traditional cuff-type sphygmomanometer. However, the user is unlikely to learn whether the heart is in a normal state. The advanced sphygmomanometer may transmit the physiological information to the cloud or an external electronic device in a wireless way. However, it is more expensive.

As to the other related technologies, a Taiwan patent of Publication No. 201503872A discloses an oscillation-type sphygmomanometer, wherein a pressure sensor detects and records the characteristics of the pressure inside the cuff and then finds out the parameters to adjust the ratio of the systolic blood pressure and the diastolic blood pressure. A Taiwan patent of No. 341115 discloses a pressure-releasing device installed in the internal circuit board inside a sphygmomanometer.

### SUMMARY OF THE INVENTION

An artificial-intelligence (AI) heart monitoring module adaptive to sphygmomanometer is provided to be incorporated into a cuff-type blood pressure monitor. The AI heart monitoring module adaptive to sphygmomanometer may joined the cuff and the blood pressure monitor with the tube which is originally connected, whereby the AI heart monitoring module adaptive to sphygmomanometer can wirelessly transmit the data of measured pressure pulses to an external device for further processing. While using a traditional cuff-type sphygmomanometer, the user may simultaneously use the AI heart monitoring module adaptive to sphygmomanometer to process and analyze the pressure wave data to obtain the information of the heart status and then transmit the information. Hence, the application of the cuff-type is expanded.

Accordingly, an AI heart monitoring module adaptive to sphygmomanometer is provided to include: a housing, including a first port and a second port thereon; a pressure sensing unit, disposed insider the housing to receive the gas passing through the first port and the second port to generate pressure-pulse data; a processing unit, disposed inside the housing, electrically connected with the pressure sensing unit, receiving and processing the pressure-pulse data coming from the pressure sensing unit; a transmission unit, disposed inside housing, and outputting all or a portion of the pressure-pulse data to the external; and an indication unit, disposed on the housing, electrically connected with the transmission unit, and responding to completion of the pressure-pulse data transmission performed by the transmission unit.

### BRIEF DESCRIPTION OF THE DRAWINGS

FIG. 1 is a block diagram schematically showing the system of an AI heart monitoring module adaptive to sphygmomanometer according to a first embodiment of the present invention.

FIG. 2 is a diagram schematically showing a scenario with application of the AI heart monitoring module adaptive to sphygmomanometer according to one embodiment of the present invention.

FIG. 3 is a front view schematically showing the appearance of the AI heart monitoring module adaptive to sphygmomanometer according to the first embodiment of the present invention.

FIG. 4 is a bottom view schematically showing the appearance of the AI heart monitoring module adaptive to sphygmomanometer according to the first embodiment of the present invention.

FIG. 5 is a diagram schematically showing a first embodiment of a display interface of the application software cooperating with the AI heart monitoring module adaptive to sphygmomanometer.

FIG. 6 is a diagram schematically showing a second embodiment of a display interface of the application software cooperating with the AI heart monitoring module adaptive to sphygmomanometer.

FIG. 7 is a diagram schematically showing a display interface which shows the analysis results obtained by the cooperation of the AI heart monitoring module adaptive to sphygmomanometer and the cloud AI according to one embodiment of the present invention.

### DESCRIPTION OF THE PREFERRED EMBODIMENT

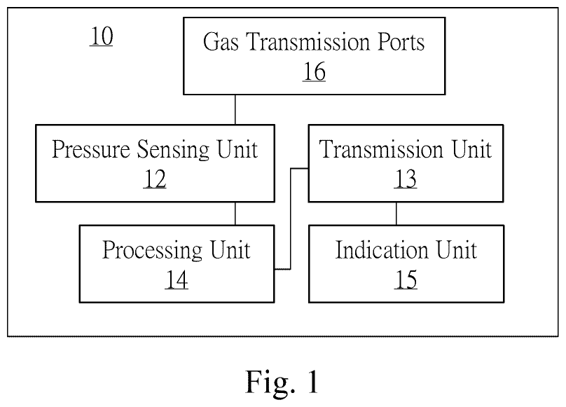

FIG. 1 is a block diagram schematically showing the system of an artificial-intelligence (AI) heart monitoring module adaptive to sphygmomanometer according to a first embodiment of the present invention. Refer to FIG. 1. The AI heart monitoring module adaptive to sphygmomanometer 10 includes a pressure sensing unit 12, a processing unit 14, a transmission unit 13 and an indication unit 15. In appearance, there is a housing encasing the pressure sensing unit 12 and the processing unit 14, and the indication unit 15 may be disposed on the housing. Gas transmission ports 16 are deposited on the housing, which include two ports configured to be jointed with gas tubes, whereby the gas inside the tubes may pass through the AI heart monitoring module adaptive to sphygmomanometer 10 via the gas transmission ports 16. During the gas inside the tube passing through the AI heart monitoring module adaptive to sphygmomanometer 10, the pressure sensing unit 12 detects the pressure of the gas passing through the AI heart monitoring module adaptive to sphygmomanometer 10 and transmits the analog data of the pressure pulses to the processing unit 14. The processing unit 14 receives and processes the received analog data of the pressure pulses (may further convert the analog data into digital data or further include processing the digital data with artificial intelligence), and then determines to transmit all or a portion of the digital data of the pressure pulses to the transmission unit 13. The transmission unit 13 transmits the data of the pressure pulses from the processing unit 14 to a device outside the AI heart monitoring module adaptive to sphygmomanometer 10, in a wire method or a wireless method, such as a Bluetooth technology or a near-field communication technology. Then, the data of the transmitted pressure pulses may be further processed, calculated and analyzed to obtain an analysis result for the status of the heart. On the other side, after the complement of transmission for the data of the pressure pulses, the transmission unit 13 may output a complement instruction to the indication unit 15 for indicating the complement of the data transmission in an appropriate way. It is understood: the AI heart monitoring module adaptive to sphygmomanometer 10 may further include a power source unit or a power supply unit (they are not shown in the drawings) to supply power to the pressure sensing unit 12, the processing unit 14, the transmission unit 13 and the indication unit 15.

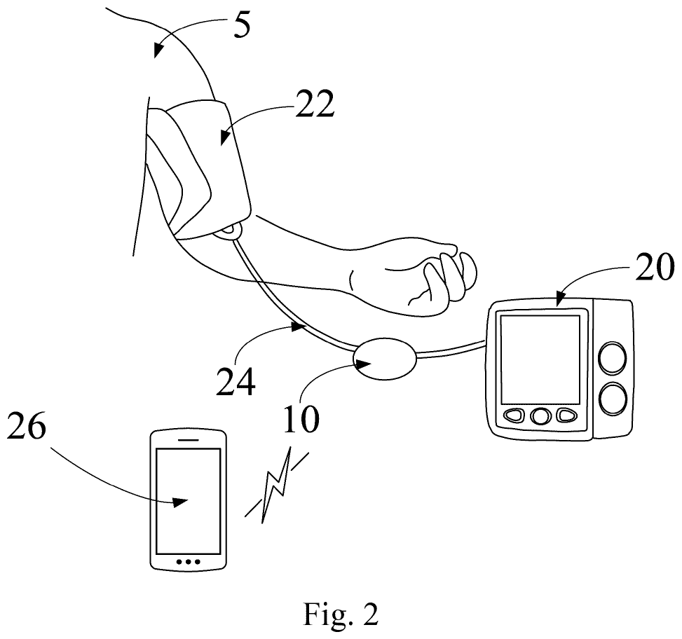

FIG. 2 is a diagram schematically showing a scenario of using the AI heart monitoring module adaptive to sphygmomanometer according to one embodiment of the present invention. Refer to FIG. 1 and FIG. 2 simultaneously. The AI heart monitoring module adaptive to sphygmomanometer 10 of the present invention may be applied to a traditional cuff-type sphygmomanometer. A gas tube 24 is connected with a sphygmomanometer 20 and a cuff 22, and the cuff 22 is fixed onto an arm of a user 5. In the embodiment, the gas tube 24 may be installed on the AI heart monitoring module adaptive to sphygmomanometer 10, and the measurement of the AI heart monitoring module adaptive to sphygmomanometer 10 does not interfere with the usage and operation of the sphygmomanometer 20 by the user 5. During the period that the user 5 or another person turns on the sphygmomanometer 20 to inflate the cuff 22 and pressurize the arm, stop inflating the cuff 22 and then release the gas, the AI heart monitoring module adaptive to sphygmomanometer 10 may detect the pressure of the gas flowing through the gas tube 24 and record the analog data of the sensed pressure pulses. Then, the AI heart monitoring module adaptive to sphygmomanometer 10 transmits all or a portion of the data of the pressure pulses to an external device 26, such as a smart phone, for succeeding processing. The AI heart monitoring module adaptive to sphygmomanometer 10 may also process the data of the pressure pulses with artificial intelligence and then transmit the results to the external device 26.

Refer to FIG. 1 and FIG. 2 again. In the scenario of using the AI heart monitoring module adaptive to sphygmomanometer 10, the measurement task of the AI heart monitoring module adaptive to sphygmomanometer 10 does not influence the regular measurement of blood pressure or other vital signs done by the sphygmomanometer 20. The AI heart monitoring module adaptive to sphygmomanometer 10 may transmit the data of the pressure pulses and heartbeats to the external device 26 for further processing. Accordingly, the AI heart monitoring module adaptive to sphygmomanometer of the present invention may overcome the drawbacks of some traditional sphygmomanometers, i.e. some traditional sphygmomanometers cannot output the measured vital sign values (such the blood pressure values). It is not good for a use to drop a traditional sphygmomanometer just because it is a device to display and record the measurement results in a closed way.

Refer to FIG. 1 and FIG. 2 again. In order to overcome the abovementioned problem, the present invention provides a simple and convenient solution: the AI heart monitoring module adaptive to sphygmomanometer 10 may be easily mounted onto and dismounted from the traditional cuff-type sphygmomanometer in a plug-and-play way, whereby the measured data may be sent out to let the user learn the analysis result about the heart. k In other words, the user may obtain the heart-related analysis result via the cooperation of the traditional cuff-type sphygmomanometer and the AI heart monitoring module adaptive to sphygmomanometer 10 of the present invention. For the people who live in rural areas or have lower income, the present invention may provide a convenient and inexpensive tool to obtain the heart-related analysis result via merely incorporating the existing cuff-type sphygmomanometer with the AI heart monitoring module adaptive to sphygmomanometer 10 of present invention. The AI heart monitoring module adaptive to sphygmomanometer 10 of the present invention can be mounted and dismounted easily in a standardized manner and will not be influenced by the variation or upgrade of the connection interface. After the present invention has completed a measurement in cooperation with a cuff-type sphygmomanometer, the present invention can be dismounted from the cuff-type sphygmomanometer and mounted to another cuff-type sphygmomanometer. Therefore, the present invention is neither limited to applying to a specified sphygmomanometer nor limited to using a specified connection method.

Refer to FIG. 1 and FIG. 2 again. The external device 26, which receives the digital data of the pressure pulses, may further process and calculate the data of the pressure pulses to obtain vital sign data, such as blood pressures and heartbeats. Alternatively, the external device 26 may transmit the data of the pressure pulses to a far-end server or an electronic device through the network, such as a cloud server or an electronic device capable to perform computation/analysis with AI. Then, the external device 26 processes and analyzes the data and stores the analysis results. The AI heart monitoring module adaptive to sphygmomanometer 10 of the present invention can transmit the data of the pressure pulses to the external device 26 for further processing the transmitted data, whereby is overcome the problem that the traditional sphygmomanometer 20 has limited computation and storage capability. It is easily understood: if the AI heart monitoring module adaptive to sphygmomanometer 10 of the present invention adopt a more powerful processing unit, the processing unit may cooperate with AI computation/analysis to analyze the data of the pressure pulses.

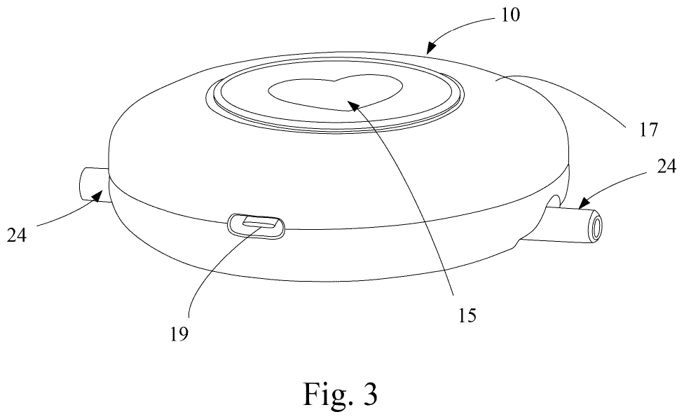

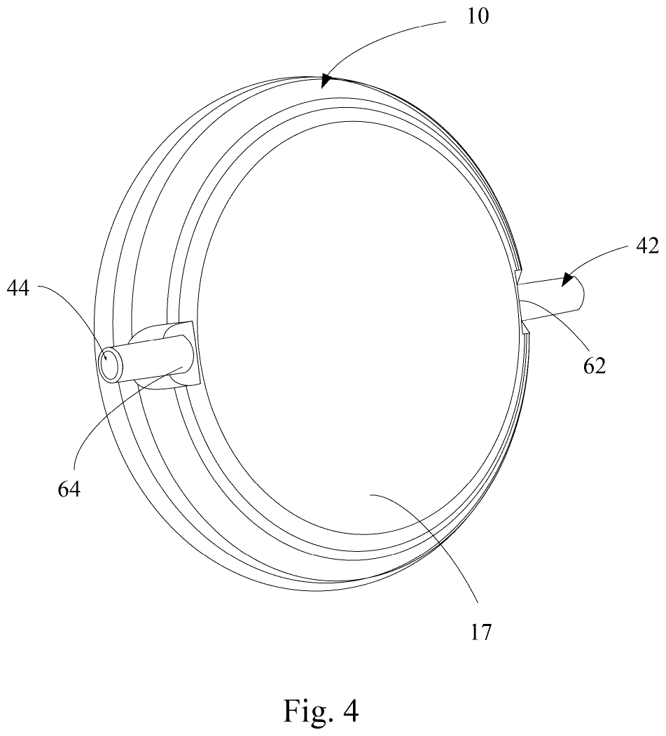

FIG. 3 is a front view schematically showing the appearance of the AI heart monitoring module adaptive to sphygmomanometer according to the first embodiment of the present invention. FIG. 4 is a bottom view schematically showing the appearance of the AI heart monitoring module adaptive to sphygmomanometer according to the first embodiment of the present invention. Refer to FIGS. 1-4 simultaneously. The AI heart monitoring module adaptive to sphygmomanometer 10 includes a housing 17 encasing the pressure sensing unit 12 and the processing unit 14. The indication unit 15 is disposed on the housing 17. The gas transmission ports 16 include two gas tube connection ports, such as a first connection port 62 and a second connection port 64. The gas tube 24 includes a first tube segment 42 and a second tube segment 44. The first tube segment 42 is disposed between and connected with the first connection port 62 and the sphygmomanometer 20. The second tube segment 44 is disposed between and connected with the second connection port 64 and the cuff 22. The AI heart monitoring module adaptive to sphygmomanometer 10 interconnects the first tube segment 42 and the second tube segment 44. Therefore, gas can flow between the first tube segment 42 and the second tube segment 44 without any retard. The abovementioned connection method is to exemplify the relationship of the gas tube and the AI heart monitoring module adaptive to sphygmomanometer of the present invention. The connection method of the present invention is not limited by the abovementioned embodiment.

Refer to FIGS. 1-4 again. The housing 17 may have an electronic connection port 19, such as a USB connection port or a Type C connection port. The AI heart monitoring module adaptive to sphygmomanometer 10 may be charged through the electronic connection port 19. In the embodiment, the pressure sensing unit 12 may be realized with different measurement technologies, such as a capacitive measurement technology, an inductance measurement technology, an optical measurement technology, a piezoelectric measurement technology, a potential measurement technology, a harmonic vibration measurement technology, or a strain measurement technology. Therefore, the pressure sensing unit 12 may be a diaphragm sensor, a solid-state sensor, a strain sensor, or a membrane sensor, etc. The processing unit 14 may be a microprocessor or an application specific integrated circuit (ASIC). The transmission unit 13 may be a transmitter transmitting information in the Bluetooth technology, the WiFi technology, the GPRS technology, or at least two thereof. The indication unit 15 may be a LED/OLED light or a buzzer.

Refer to FIGS. 1-4 again. In another embodiment, during the sphygmomanometer 20 inflates the cuff 22 to pressurize the arm, stops inflating and releases gas, the AI heart monitoring module adaptive to sphygmomanometer 10 may sense the analog signals of the pressure pluses, measure the variation of the pressure pulses, convert the analog data of the pressure pulses into digital data of the pressure pulses, and analyze the digital data of the pressure pulses. At the moment that the sphygmomanometer 20 stops inflating the cuff 22, the AI heart monitoring module adaptive to sphygmomanometer 10 outputs all or a portion of the data of the digital data of the pressure pulses to the external device 26. After the transmission of the data of the pressure pulses is completed, the AI heart monitoring module adaptive to sphygmomanometer 10 transmits an instruction to the indication unit 15 to inform the user that the transmission of the data of the pressure pulses has been completed. It is easily understood: the indication unit 15 may be programmed to synchronously indicate that the transmission of the data of the pressure pulses is being performed. Appropriate application software (APP) may be installed in the external device 26, such as a smart phone, to display the data of the pressure pulses on the screen of the smart phone. It is an option: APP may calculate the data of pressure pulses to obtain the corresponding vital sign information through the computation capability of the smart phone. It is another option: the far-end server uses AI to process and analyze the data of the pressure pulses to obtain the corresponding vital sign information, and the smart phone receives the corresponding vital sign information from the far-end server and displays the information on the screen. The vital sign information displayed by the indication unit 15 may include pulses, heartbeats, and the status of the heart.

According to the above description, the AI heart monitoring module adaptive to sphygmomanometer 10 of the present invention is applicable to the traditional cuff-type sphygmomanometer that has fewer functions. The user needn't spend additional money on buying an expensive sphygmomanometer but can use the AI heart monitoring module adaptive to sphygmomanometer 10 to transmit the measured data to an external device. In the environment that almost everyone has a smart phone, the user may use his smart phone to communicate with the AI heart monitoring module adaptive to sphygmomanometer 10 to obtain the measured data and perform analysis to obtain the corresponding vital sign information.

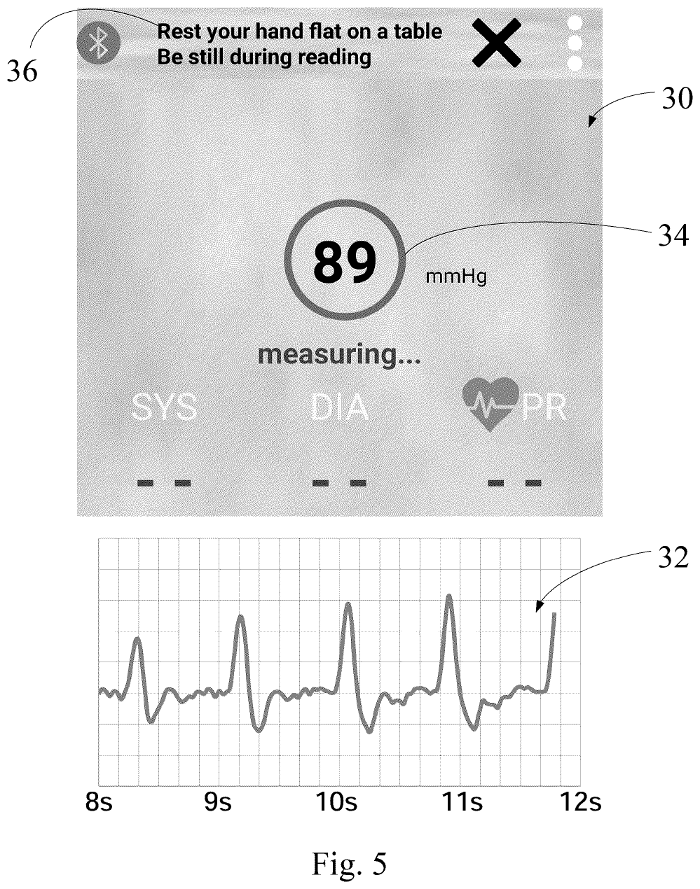

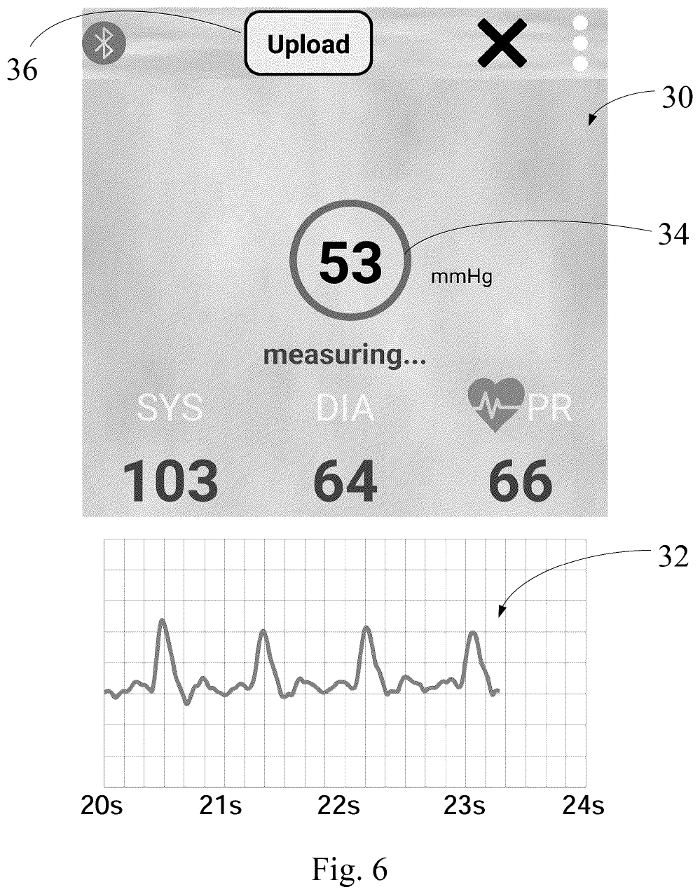

FIG. 5 is a diagram schematically showing a first embodiment of a display interface of the application software cooperating with the AI heart monitoring module adaptive to sphygmomanometer. FIG. 6 is a diagram schematically showing a second embodiment of a display interface of the application software cooperating with the AI heart monitoring module adaptive to sphygmomanometer. Refer to FIGS. 1-6. The traditional cuff-type sphygmomanometer only has limited functions. The AI heart monitoring module adaptive to sphygmomanometer 10 of the present invention cooperates with a piece of application software to display the status of measurement through the external device 26 which may be a smart phone used nowadays. The user may use the external device 26 to start the corresponding application software to open a screen image 30. During the process that the user uses the sphygmomanometer 20 and the cuff 22 to perform gas inflation and gas release, the AI heart monitoring module adaptive to sphygmomanometer 10 measures the real-time pressure pulses and heartbeat pulses and transmits the measurement results to the external device 26. Then, the external device 26 presents the measurement results, such as the blood pressure value 34 and the heartbeat pulse graph 32, on the screen image 30. The screen image 30 may further include other indications and records; for example, the screen image 30 has one or more indication areas 36, including an area to remind the user of his posture for measurement; an area to indicate that the cuff is being inflated or deflated; an area to remind the user that blood pressure is being measured; an area to remind the user that the pressure pulse and the heartbeat are being transmitted to the cloud. However, the present invention is not limited by the abovementioned embodiments.

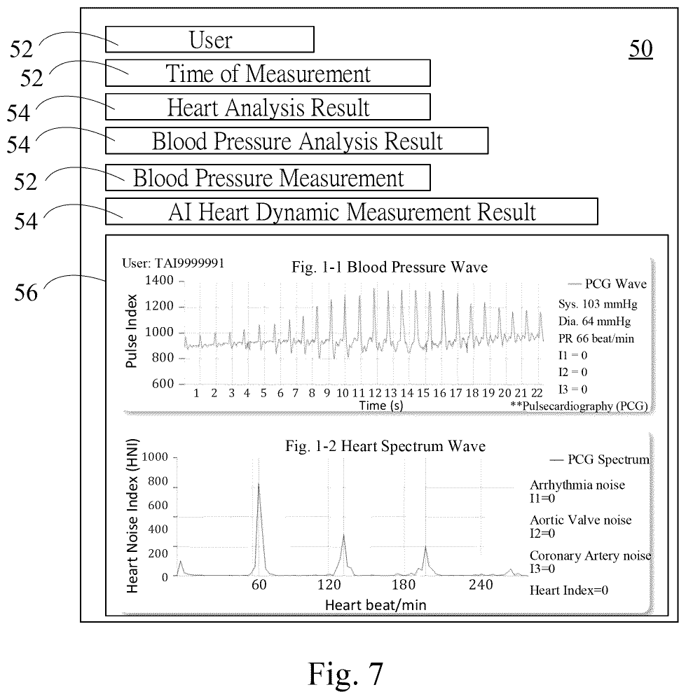

FIG. 7 is a diagram schematically showing a display interface which shows the analysis results obtained by the cooperation of the AI heart monitoring module adaptive to sphygmomanometer and the cloud AI according to one embodiment of the present invention. Refer to FIGS. 1-7. The external device 26 receives the measured data from the AI heart monitoring module adaptive to sphygmomanometer 10 and presents the data on the screen image 30. At the same time, the AI heart monitoring module adaptive to sphygmomanometer 10 transmits one or more of pieces of data of pressure pluses and one or more of pieces of data of heartbeat pulses to a cloud server in the network for analysis through a wired method or a wireless method (such as the WiFi technology). In one embodiment, the cloud server includes AI-based computation/analysis programs/software, which may process and analyze the data of pressure pluses and the data of heartbeat pulses, which have been transmitted to the cloud server, to obtain the corresponding information of the blood pressure and the heart of the user. For example, after the cloud server obtains the results of analyzing the data of pressure pluses and the data of heartbeat pulses, the user is informed to use the external device 26 to access the Internet and look up a blood pressure/heart monitoring list 50. The blood pressure/heart monitoring list 50 may include but is not limited to one or more basic measurement data display areas 52, one or more analysis result display areas 54, and one or more measurement/analysis graph display areas 56. The data displayed by the basic measurement data display areas 52 include user's data (including the user's name and the user's code) and measurement data (including the measurement time and the measured blood pressure). The graphs displayed by the measurement/analysis graph display areas 56 include the graph of the blood pressure pulses and the graph of the heartbeat spectrum. The analysis result display areas 54 display the information obtained via analyzing the data of pressure pulses and the data of the heartbeats; for example, the information includes “the analysis result of the heart is normal”, “the analysis result of the blood pressure is normal”, and “the analysis result of heartbeat is normal”. It is an option: the vital sign information displayed on the analysis result display areas 54 may also be displayed on the indication unit of the AI heart monitoring module adaptive to sphygmomanometer 10. It is an option: the AI-based computation/analysis program/software may be directly installed in the AI heart monitoring module adaptive to sphygmomanometer.

The embodiments described above are only to demonstrate the technical thoughts and characteristics of the present invention to enable the persons skilled in the art to understand, make, and use the present invention. However, it is not intended to limit the scope of the present invention. Any equivalent modification or variation according to the spirit of the present invention is to be also included by the scope of the present invention.

What is claimed is:

1. An artificial-intelligence heart monitoring module adaptive to sphygmomanometer, comprising
a housing having a first connection port and a second connection port;
a pressure sensing unit, disposed inside the housing, sensing pressure pulses of gas passing through the first connection port and the second connection port to obtain analog data of the pressure pulses;
a processing unit, disposed inside the housing, electrically connected with the pressure sensing unit, receiving the analog data of the pressure pulses, and converting the analog data of the pressure pulses into digital data of the pressure pulses;
a transmission unit, disposed inside the housing, electrically connected with the processing unit, and outputting all or a portion of the digital data of the pressure pulses to an external device; and
an indication unit, disposed on the housing, electrically connected with the transmission unit, and responding to an event that the transmission unit completes outputting the digital data of the pressure pulses to the external device.

2. The artificial-intelligence heart monitoring module adaptive to sphygmomanometer according to claim 1, wherein the first connection port and the second connection port are respectively connected with a first gas tube segment and a second gas tube segment, which are disposed between a cuff and a sphygmomanometer; the sphygmomanometer outputs gas to inflate the cuff through the first gas tube segment and the second gas tube segment.

3. The artificial-intelligence heart monitoring module adaptive to sphygmomanometer according to claim 1, wherein the pressure sensing unit is a diaphragm sensor, a solid-state sensor, a strain sensor, or a membrane sensor.

4. The artificial-intelligence heart monitoring module adaptive to sphygmomanometer according to claim 1, wherein the processing unit includes a microprocessor.

5. The artificial-intelligence heart monitoring module adaptive to sphygmomanometer according to claim 1, wherein the transmission unit is a transmitter transmitting information in a Bluetooth technology, a WiFi technology, a GPRS technology, or at least two thereof.

6. The artificial-intelligence heart monitoring module adaptive to sphygmomanometer according to claim 1, wherein the indication unit is a LED light, an OLED light or a buzzer.

7. The artificial-intelligence heart monitoring module adaptive to sphygmomanometer according to claim 1, further comprising an electronic connection port disposed on the housing.

8. The artificial-intelligence heart monitoring module adaptive to sphygmomanometer according to claim 1, wherein the digital data of the pressure pulses is output to the external device and processed by the external device with artificial intelligence to obtain a plurality of pieces of vital sign information; the indication unit displays the plurality of pieces of vital sign information.

9. An application program, cooperating with the artificial-intelligence heart monitoring module adaptive to sphygmomanometer according to claim 1, comprising functions of outputting the digital data of the pressure pulses and displaying the digital data of the pressure pulses.

10. The application program according to claim 9, wherein the digital data of the pressure pulses is output to the external device and processed by the external device to obtain a plurality of pieces of vital sign information; the application program includes functions of receiving the plurality of pieces of vital signal information and displaying the plurality of pieces of vital signal information.
